(19) United States
(12) Patent Application Publication
(10) Pub. No.: US 2010/0093646 A1
(43) Pub. Date: Apr. 15, 2010

(54) METHOD FOR ENHANCING MEMORY IN NORMAL INDIVIDUAL

(75) Inventors: Yasushi Sakai, Tsukuba-shi (JP); Takeshi Ikeda, Tsukuba-shi (JP); Atsumi Nitta, Nagoya-shi (JP); Yoko Hibi, Nagoya-shi (JP)

Correspondence Address: SUGHRUE MION, PLLC, 2100 PENNSYLVANIA AVENUE, N.W., SUITE 800, WASHINGTON, DC, 20037, US

(73) Assignees: KYOWA HAKKO BIO CO., LTD., Tokyo (JP); NATIONAL UNIVERSITY CORPORATION NAGOYA UNIVERSITY, Nagoya-shi (JP)

(21) Appl. No.: 12/571,084

(22) Filed: Sep. 30, 2009

Related U.S. Application Data
(63) Continuation-in-part of application No. 12/446,893, filed on Apr. 23, 2009.

(30) Foreign Application Priority Data
Oct. 23, 2006 (JP) 2006-287639
Oct. 23, 2007 (JP) PCT/JP2007/070628

(51) Int. Cl. A61K 38/05; A61P 25/00

(52) U.S. Cl. 514/19

(57) ABSTRACT

The present invention provides a method for enhancing memory in a normal individual, particularly in an individual of middle or advanced age who is regarded as clinically normal. The method for enhancing memory in a normal individual comprises administration of a peptide consisting of Leu and Ile or a modified form thereof.

### CROSS-REFERENCE TO RELATED APPLICATIONS

This application is a continuation-in-part of U.S. patent application Ser. No. 12/446,893 filed on Oct. 23, 2007. The entire contents of all of the above-identified application are hereby incorporated by reference.

### BACKGROUND OF THE INVENTION

The present invention relates to a method for enhancing memory in a normal individual, particularly in an individual of middle or advanced age who is regarded as clinically normal, and a novel memory enhancer that is effective for retention and improvement of memory in such normal individuals.

In association with population aging because of improved living standards or recent progress in medicine, memory loss including mild forgetfulness is becoming a major issue of concern in the society not only for patients with dementia (e.g. dementia of the Alzheimer type) exhibiting serious symptoms, but also for individuals of middle or advanced age who are regarded as clinically normal. Meanwhile, since early education of teenaged students or pupils is remarkably intensifying, memory improvement is a major issue of concern for young individuals as well. Thus, it is deduced that retention and improvement of memory will become more and more important for normal individuals of middle or advanced age or young individuals in the future. In spite of the existence of such needs, modern medicine does not provide any measures as in a treatment of diseases such as dementia of the Alzheimer type, and development of various measures including functional food has been awaited.

It has been known that a dipeptide Leu-Ile has therapeutic effects on drug dependence (Nitta A. et al., J. Neurosci. Res., 78: 250-258 (2004) and Nitta A. et al., Folia Pharmacologica Japonica, 122: 81-83 (2003)) and it has effects of activating Akt associated with the glial cell line-derived neurotrophic factor (GDNF) that suppresses drug dependence (WO 2006/090555). However, the fact that dipeptide Leu-Ile has memory-enhancing effects has previously been unknown.

### SUMMARY OF THE INVENTION

The present invention is intended to provide a method for enhancing memory in a normal individual, particularly in an individual of middle or advanced age who is regarded as clinically normal. Also, the present invention is intended to provide a novel memory enhancer that is effective for retention and improvement of memory in a normal individual, particularly in an individual of middle or advanced age who is regarded as clinically normal.

In order to attain the above objects, the present inventors have conducted concentrated studies regarding pharmacological effects of a dipeptide Leu-Ile on normal animals. As a result, they discovered that the dipeptide Leu-Ile has significant memory-enhancing effects, thereby completing the present invention.

The present invention has been completed based on the above finding and includes the following inventions.

(1) A method for enhancing memory in a normal individual, which comprises administering a peptide consisting of Leu and Ile or a modified form thereof.

(2) The method according to (1), which comprises administering Leu-Ile or a modified form of Leu-Ile.

(3) The method according to (1) which comprises administering Leu-Ile.

(4) A memory enhancer comprising a peptide consisting of Leu and Ile or a modified form thereof as an active ingredient.

(5) The memory enhancer according to (4) comprising Leu-Ile or a modified form of Leu-Ile as an active ingredient.

(6) The memory enhancer according to (4) comprising Leu-Ile as an active ingredient.

(7) Use of a peptide consisting of Leu and Ile or a modified form thereof in the production of a memory enhancer.

(8) The use according to (7), wherein the peptide consisting of Leu and Ile or a modified form thereof is Leu-Ile or a modified form of Leu-Ile.

(9) The use according to (7), wherein the peptide consisting of Leu and Ile or a modified form thereof is Leu-Ile.

In this description, a peptide sequence is shown to have an amino terminus at the left end and a carboxyl terminus at the right end in accordance with a conventional method. When an amino acid residue is of an L-form, indication thereof is omitted.

The present invention provides a method for enhancing memory in a normal individual, particularly in an individual of middle or advanced age who is regarded as clinically normal. Also, the present invention provides a novel memory enhancer that is effective for retention and improvement of memory for a normal individual, particularly in an individual of middle or advanced age who is regarded as clinically normal.

### BRIEF DESCRIPTION OF THE DRAWINGS

FIG. 1 shows a chart showing the results of recognition index measurement using the novel object recognition test of Example 1.

FIG. 2 shows a chart showing the results of measuring the latency of drinking using the water-finding test of Example 2.

FIG. 3 shows a chart showing the results of recognition index measurement using the novel object recognition test of Example 3.

### DETAILED DESCRIPTION OF THE INVENTION

In the present invention, examples of a peptide consisting of Leu and Ile include Leu-Ile and Ile-Leu, with the use of Leu-Ile being preferable.

In the present invention, the term “a peptide consisting of Leu and Ile or a modified form thereof” refers to a compound having a structure that is at least partially different from a basic structure consisting of Leu and Ile (i.e., dipeptide), via modification of a part (and possibly a plurality of sites) of the basic structure, such as substitution with another atomic group or addition of another molecule. Further, a dipeptide in which at least one of Leu and Ile is converted into a D- or DL-form is within the scope of the peptide consisting of Leu and Ile or a modified form thereof.

Representative examples of modified forms in the present invention include peptide derivatives in which part of the Leu or Ile side chain has been substituted with another atom or a group of atoms. Examples of such atom or a group of atoms include hydroxyl groups, halogens (e.g., fluorine, chlorine, bromine, and iodine), alkyl groups (e.g., methyl, ethyl, n-propyl, and isopropyl groups), hydroxyalkyl groups (e.g., hydroxymethyl and hydroxyethyl groups), alkoxy groups (e.g., methoxy and ethoxy groups), and acyl groups (e.g., formyl, acetyl, malonyl, and benzoyl groups).

The modified peptide of the present invention includes a modified peptide in which a functional group in Leu or Ile is protected by an adequate protecting group. Examples of protecting groups that can be used for such purpose include acyl groups, alkyl groups, monosaccharides, oligosaccharides, and polysaccharides. Such a protecting group is bound to a peptide via an amide bond, an ester bond, a urethane bond, a urea bond, or the like. It depends on, for example, a peptide site to which a protecting group is to be bound or a type of a protecting group to be used.

Other examples of the modified peptide of the present invention include a modified peptide in which a sugar chain is added. Also, examples of the modified peptide of the present invention include various peptide derivatives classified as alkylamines, alkylamides, sulfinyls, sulfonylamides, halides, amides, amino alcohols, esters, or aminoaldehydes, wherein the N- or C-terminus is substituted with another atom or the like. A peptide derivatives composed by combining the above-explained various modification techniques may also be examples of the modified peptide of the present invention.

The peptides consisting of Leu and Ile or a modified form thereof according to the present invention include salts of the above peptides, salts of the modified peptides, and hydrates thereof. Salt used in the present invention is not particularly limited, provided that such salt is pharmaceutically acceptable. Examples thereof include salts with hydrochloric acid, phosphoric acid, sulfuric acid, nitric acid, and boric acid (i.e., inorganic acid salts) and salts with formic acid, acetic acid, lactic acid, fumaric acid, maleic acid, tartaric acid, and citric acid (i.e., organic acid salts). Such salts can be prepared in accordance with a conventional technique.

In the present invention, examples of memory enhancer include pharmaceutical preparations and dietary supplements. Examples of dietary supplements include oral preparations that have similar configurations as pharmaceutical preparations but are not approved as pharmaceutical preparations by the Pharmaceutical Affairs Law. Examples also include food products to which the active ingredient of the present invention has been added.

Specifically, the present invention relates to a pharmaceutical composition and a nutritional composition used for dietary supplements which comprise a peptide consisting of Leu and Ile or a modified form thereof as an active ingredient.

The term “pharmaceutical preparation” in the present invention refers to a preparation prepared by adding a carrier that is generally used as a base for pharmaceutical preparation, such as an excipient, a disintegrator, a lubricant, a buffering agent, a binder, an emulsifier, a suspension, a soothing agent, a stabilizer, a preservative, an antiseptic, a physiological saline solution, or the like, to an active ingredient of the present invention. Examples of formulations include tablets, powders, fine grains, granules, capsules, syrups, injections, external preparations, and suppositories. Examples of excipients that can be used include lactose, starch, sorbitol, D-mannitol, and saccharose. Examples of disintegrators that can be used include starch, carboxymethylcellulose, and calcium carbonate. Examples of buffers that can be used include phosphate, citrate, and acetate. Examples of emulsifying agents that can be used include gum Arabic, sodium alginate, and gum tragacanth. Examples of binders that can be used include Pullulan, gum Arabic, gelatin, and starch. Examples of lubricants that can be used include magnesium stearate, methylcellulose, and magnesium silicate. Examples of suspensions that can be used include glyceryl monostearate, aluminum monostearate, methylcellulose, carboxymethylcellulose, hydroxymethylcellulose, and sodium lauryl sulfate. Examples of soothing agents that can be used include benzyl alcohol, chlorobutanol, and sorbitol. Examples of stabilizers that can be used include propylene glycol, diethylin sulfite, and ascorbic acid. Examples of preservatives that can be used include phenol, benzalkonium chloride, benzyl alcohol, chlorobutanol, and methylparaben. Examples of antiseptic agents that can be used include benzalkonium chloride, parahydroxybenzoic acid, and chlorobutanol.

In the present invention, the term “oral dietary supplements” refers to a preparation prepared by adding a carrier that is generally used as a nutritional food preparation base, such as an excipient, a disintegrator, an emulsifier, a stabilizer, a lubricant, a buffering agent, a flavoring agent, or the like, to an active ingredient of the present invention. Examples of formulations include tablets, powders, fine granules, granules, and capsules.

Examples of dietary supplements prepared by adding the active ingredient of the present invention to food include nutritional beverages, soft drinks, and jelly produced by a conventional technique with the use of the active ingredient of the present invention.

A peptide consisting of Leu and Ile can be prepared by a known method of peptide synthesis (e.g., a solid-phase synthesis method and a liquid-phase synthesis method). When the peptide of the present invention or the like exists in nature, such peptide can be obtained via operation such as extraction or purification. Examples of sources for the peptide of the present invention or the like include animal cells (including those of humans), plant cells, and body fluids (e.g., blood and urine).

Further, an efficient method for producing a dipeptide using microorganisms, enzymes, or the like is known (WO 2004-058960), and a peptide consisting of Leu and Ile can be efficiently prepared in accordance with the production method disclosed in the above publication.

Furthermore, a modified form of the peptide consisting of Leu and Ile can be prepared in accordance with a conventional technique as described above.

A pharmaceutical preparation or oral dietary supplements comprising, as an active ingredient, a modified form of the peptide consisting of Leu and Ile can be prepared in accordance with a conventional technique using a modified form of the peptide consisting of Leu and Ile as a main active ingredient.

A pharmaceutical preparation of interest can be produced by incorporating other pharmaceutically acceptable ingredients, such as a carrier, an excipient, a disintegrator, a buffer, an emulsifying agent, a binder, a lubricant, a suspension agent, a soothing agent, a stabilizer, a preservative, an antiseptic agent, or physiological saline, into a modified form of a peptide consisting of Leu and Ile. Examples of excipients that can be used include lactose, starch, sorbitol, D-mannitol, and saccharose. Examples of disintegrators that can be used include starch, carboxymethylcellulose, and calcium carbonate. Examples of buffers that can be used include phosphate, citrate, and acetate. Examples of emulsifying agents that can be used include gum Arabic, sodium alginate, and gum tragacanth. Examples of binders that can be used include Pullulan, gum Arabic, gelatin, and starch. Examples of lubricants that can be used include magnesium stearate, methylcellulose, and magnesium silicate. Examples of suspensions that can be used include glyceryl monostearate, aluminum monostearate, methylcellulose, carboxymethylcellulose, hydroxymethylcellulose, and sodium lauryl sulfate. Examples of soothing agents that can be used include benzyl alcohol, chlorobutanol, and sorbitol. Examples of stabilizers that can be used include propylene glycol, diethylin sulfite, and ascorbic acid. Examples of preservatives that can be used include phenol, benzalkonium chloride, benzyl alcohol, chlorobutanol, and methylparaben. Examples of antiseptic agents that can be used include benzalkonium chloride, parahydroxybenzoic acid, and chlorobutanol.

With the use of such base materials for pharmaceutical preparations, formulations of interest, such as tablets, powders, fine grains, granules, capsules, syrups, injections, external preparations, and suppositories, can be prepared in accordance with a conventional technique.

Also, a modified form of a peptide consisting of Leu and Ile can be added to prepare nutritional food products, such as nutritional beverages, soft drinks, and jelly in accordance with a conventional technique.

The thus-prepared memory enhancer comprising the peptide consisting of Leu and Ile of the present invention or a modified form thereof as an active ingredient can be administered orally or parenterally (e.g., via intravenous, intraarterial, subcutaneous, intramuscular, or intraperitoneal injection) to a patient, depending on the form thereof. In general, the content of the active ingredient (e.g., peptide) in the agent of the present invention varies depending on formulation. In the case of a liquid formulation, such as an injection, for example, such content is about 0.001% by weight to about 10% by weight, preferably about 0.01% by weight to about 3% by weight, and particularly preferably about 0.1% by weight to about 1% by weight. In the case of a solid formulation, such as a tablet, such content is about 0.1% by weight to about 90% by weight, preferably about 1% by weight to about 50% by weight, and particularly preferably about 3% by weight to about 30% by weight.

The present invention provides a method for enhancing memory in a normal individual, particularly in an individual of middle or advanced age who is regarded as clinically normal, comprising administration of a peptide consisting of Leu and Ile or a modified form thereof. The term “normal individual” used herein preferably refers to a human who is not diagnosed as having clinical diseases or disorders. More preferably, the term refers to a human who is not clinically diagnosed to have any form of dementia including dementia of the Alzheimer type. Especially, the term refers to a human who is not diagnosed to have any form of dementia, including dementia of the Alzheimer type, Parkinson's disease, Pick's disease, and mild cognitive impairment (MCI). Also, the term “individual(s) of middle or advanced age” used herein refers to middle-aged people aged from about 40 or 50 years to about 65 years and elderly people aged about 65 years or over. The method for enhancing memory of the present invention comprises a step of administering a pharmaceutical preparation comprising, as an active ingredient, a peptide consisting of Leu and Ile or a modified form thereof to a living body. The route of administration is not particularly limited. Examples thereof include oral, intravenous, intracutaneous, subcutaneous, intramuscular, intraperitoneal, percutaneous, and transmucosal routes. The dose of agent varies depending on symptoms, age, sex, and body weight of a patient, and the like. A person skilled in the art can adequately determine a suitable dose. When a preparation comprising Leu-Ile as an active ingredient is used, for example, the dose can be determined such that the amount of an active ingredient for an adult (body weight: about 60 kg) ranges from about 0.1 to about 3,000 mg per day, preferably from about 1 mg to about 2,000 mg per day, and more preferably from about 3 mg to about 1,000 mg per day. Administration can be carried out, for example, once to several times per day, once every other day, or once every three days.

In the present invention, a method for enhancing memory in a normal individual which comprises administering a peptide consisting of Leu and Ile or a modified form thereof is that a normal individual takes a pharmaceutical preparation comprising, as an active ingredient, a peptide consisting of Leu and Ile or a modified form thereof in order to improve his/her memory, or that an individual of middle or advanced age who is regarded as clinically normal takes the same in order to prevent memory loss or ameliorate memory during the normal process of aging. When determining the administration schedule, symptoms of a patient, the duration of drug efficacy, and other conditions can be taken into consideration.

### EXAMPLES

The effects of enhancing memory of the present invention were confirmed by the novel object recognition test (Ennaceur et al., Behav Brain Res. 80, 9-25, 1996) and a water-finding test (Nabeshima et al., Meth Neurosci. 14, 217-229, 1993) as described below.

Examples of the present invention are described below.

### Example 1

### Evaluation of Memory-Enhancing Effects by Novel Object Recognition Test

Male C57BL mice (body weights: 20 g to 22 g; 6 months old) were divided into two groups (n=6 or 7), and physiological saline or Leu-Ile (150 μmol/kg) was administered subcutaneously thereto once a day over a period of 20 days. Mice were acclimated to an apparatus (30 cm (length)×30 cm (width)×35 cm (height)) in advance, 2 identical objects were placed in the apparatus immediately after administration on day 19, and mice were allowed to freely explore for the objects for 10 minutes (exploration trial). In an apparatus in which one of the objects was replaced with a novel object, mice were allowed to freely explore for the objects for 10 minutes on day 20 (24 hours after the exploration trial) (memory test trial). In the memory test trial, the proportion (%) of the time required for novel object recognition relative to the total time required for explore was designated as the recognition index, and comparison thereof was carried out between the two groups. A higher recognition index value represents a higher level of visual cognitive memory. Statistical analysis was carried out using the Student's t-test (with a level of significance of lower than 5% being evaluated as representing a significant difference) and the effects of Leu-Ile for enhancing memory were assayed.

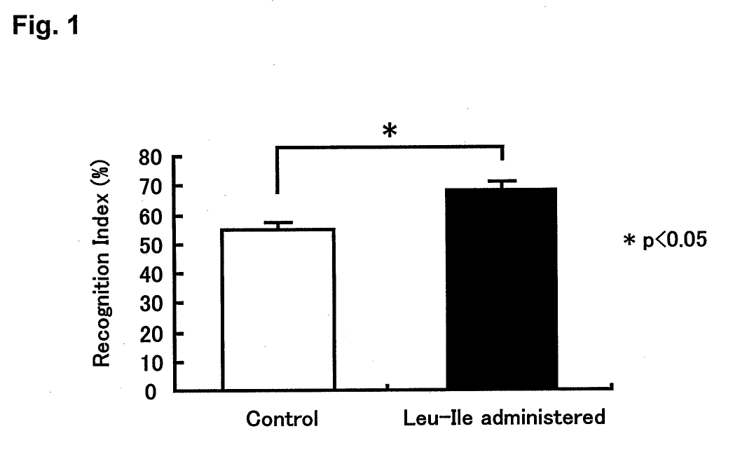

FIG. 1 shows the results of recognition index measurement. Compared with the control group, the group to which Leu-Ile had been administered exhibited a significantly higher recognition index. This demonstrates that Leu-Ile has memory-enhancing effects.

### Example 2

### Evaluation of Memory-Enhancing Effects by Water-Finding Test

Male C57BL mice (body weights: 20 g to 22 g; 6 months old) were divided into two groups (n=6 or 7), and physiological saline or Leu-Ile (150 μmol/kg) was administered subcutaneously thereto once a day over the period of 28 days. On day 25, mice were placed in an open field having small chambers equipped with water-feeding bottles, and mice were allowed to freely explore for water for 3 minutes amidst the equipment (exploration trial). After the completion of the exploration trial, mice were immediately returned to the home cage. At the same time, water deprivation was initiated. On day 26 (24 hours after the exploration trial), mice were placed at the same positions in the apparatus, they were given a chance to explore for water until they found water-feeding bottles and to drink water, and they were allowed to remember the positions of the water-feeding bottles (memory test trial 1). On the following day, mice were subjected to water deprivation again, and the memory test trial was performed again 24 hours thereafter (day 28) (memory test trial 2). The duration that was necessary until the mice drank water from the water-feeding bottles since they had been placed in the equipment in memory test trial 2 was designated as the latency of drinking, and comparison thereof was carried out between two groups. A smaller latency of drinking value represents a higher capacity for spatial memory. Statistical analysis was carried out using the Student's t-test (with a level of significance of lower than 5% being evaluated as representing a significant difference) and memory-enhancing effects of Leu-Ile were assayed.

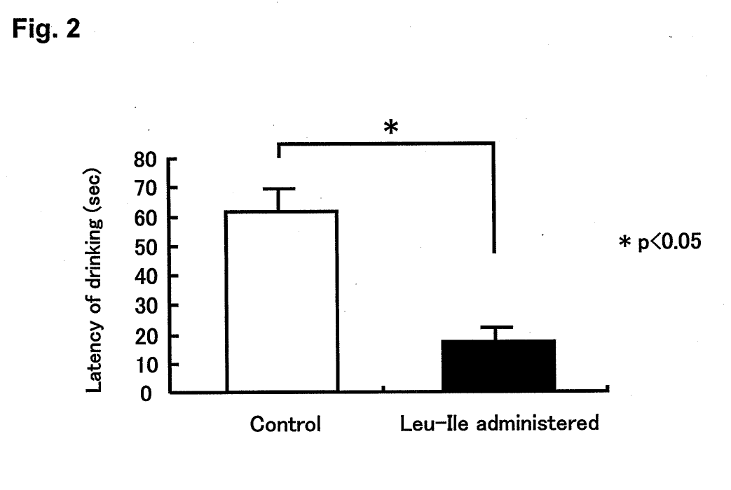

FIG. 2 shows the results of measuring the latency of drinking. As shown in FIG. 2, the group to which Leu-Ile had been administered exhibited a significantly shorter latency of drinking, compared with the control group. This indicates that Leu-Ile has memory-enhancing effects.

### Example 3

### Evaluation of Memory-Enhancing Effects by Novel Object Recognition Test (2)

Male C57BL mice (body weights: 20 g to 22 g; 7 to 12 months old) were divided into four groups (n=11 or 12), and physiological saline or Leu-Ile (50, 150, or 750 μmol/kg) was administered orally thereto once a day over a period of 31 days. Mice were acclimated to an apparatus (30 cm (length)×30 cm (width)×35 cm (height)) in advance, 2 identical objects were placed in the apparatus immediately after the administration on day 30, and mice were allowed to freely explore for the objects for 10 minutes (exploration trial). In an apparatus in which one of the objects was replaced with a novel object, mice were allowed to freely explore for the objects for 10 minutes on day 31 (24 hours after the exploration trial) (memory test trial). In the memory test trial, the proportion (%) of the time required for novel object recognition relative to the total time required for explore was designated as the recognition index and comparison thereof was carried out among the four groups. A higher recognition index value represents a higher level of visual cognitive memory. Statistical analysis was carried out via variance analysis (with a level of significance of lower than 5% being evaluated as representing a significant difference) and the memory-enhancing effects of Leu-Ile were assayed.

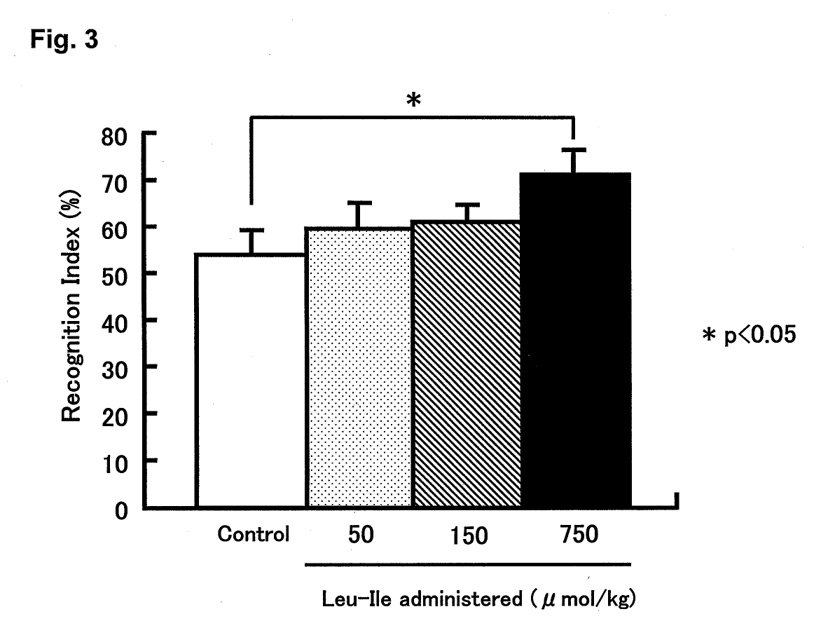

FIG. 3 shows the results of recognition index measurement. Compared with the control group, the group to which Leu-Ile had been administered exhibited a significantly higher recognition index. This demonstrates that Leu-Ile has memory-enhancing effects.

### Preparation Example 1

### Tablet

Tablets having the composition below are prepared by a conventional technique.

Formulation:

| | | |
|---|---|---|
| Leu-Ile | 25 | mg |
| Lactose | 138.4 | mg |
| Potato starch | 30 | mg |
| Hydroxypropylcellulose | 6 | mg |
| Magnesium stearate | 0.6 | mg |
| Total | 200 | mg |

### Preparation Example 2

### Injection

An injection having the composition below is prepared by a conventional technique.

Formulation:

| Leu-Ile | 2 | mg |
|---|---|---|
| D-mannitol | 10 | mg |
| Aqueous solutions of hydrochloric acid | adequate amount | |
| Aqueous solutions of sodium hydroxide | adequate amount | |
| Distilled water for injection | adequate amount | |
| Total | 2.00 | ml |

### Preparation Example 3

### Dietary Supplement Tablet

Dietary supplement tablets having the composition below are prepared by a conventional technique.

Formulation:

| | | |
|---|---|---|
| Leu-Ile | 20 | mg |
| Erythritol | 245 | mg |
| Carboxymethylcellulose calcium | 5 | mg |
| Flavoring agent | 10 | mg |
| Sucrose fatty acid ester | 20 | mg |
| Total | 300 | mg |

All publications, patents, and patent applications cited herein are incorporated herein by reference in their entirety.

The present invention is applicable in the pharmaceutical and food industries as a pharmaceutical composition or nutritional composition for dietary supplements that is intended for retention and improvement of memory in a normal individual, particularly in an individual of middle or advanced age who is regarded as clinically normal.

1. A method for enhancing memory in a normal individual, which comprises administering a peptide consisting of Leu and Ile or a modified form thereof.

2. The method according to claim 1, which comprises administering Leu-Ile or a modified form of Leu-Ile.

3. The method according to claim 1, which comprises administering Leu-Ile.
